(19) United States
(12) Patent Application Publication
(10) Pub. No.: US 2010/0240700 A1
(43) Pub. Date: Sep. 23, 2010

(54) PROPHYLACTIC PRETREATMENT WITH ANTIOXIDANTS

(75) Inventor: Kameron W. Maxwell, Vida, OR (US)

Correspondence Address: KNOBBE MARTENS OLSON & BEAR LLP, 2040 MAIN STREET, FOURTEENTH FLOOR, IRVINE, CA, 92614, US

(73) Assignee: MITOS PHARMACEUTICALS, INC., Newport Beach, CA (US)

(21) Appl. No.: 12/793,606

(22) Filed: Jun. 3, 2010

Related U.S. Application Data
(63) Continuation of application No. 10/554,299, filed on Sep. 22, 2006.
(60) Provisional application No. 60/465,909, filed on Apr. 25, 2003.

(51) Int. Cl. A61K 31/445; A61K 31/4015; A61K 31/421; A61P 9/10

(52) U.S. Cl. 514/315; 514/424; 514/374

(57) ABSTRACT

Methods, compositions, and uses for pre-treating patients who are susceptible to ischemia, including stroke, with nitroxides, in order to prevent or ameliorate the effects of stroke or other ischemic disease.

### CROSS-REFERENCE TO RELATED APPLICATIONS

The present application is a continuation of U.S. patent application Ser. No. 10/554,299, filed Sep. 22, 2006, which is a U.S. National Phase of International Application No. PCT/US2004/012640, filed Apr. 22, 2004, which claims the benefit of U.S. Provisional Application No. 60/465,909 filed Apr. 25, 2003 which are expressly incorporated by reference in their entireties.

### FIELD OF THE INVENTION

The present invention relates to methods of pre-treating patients who are susceptible to ischemia with nitroxides, in order to prevent or ameliorate the effects of ischemia.

In Western countries, strokes are the most common cause of disabling neurologic damage. In general, a stroke occurs when blood and oxygen flow to the brain is disrupted and brain damage results. While the onset of a stroke can be unpredictable, it is well known that certain medical procedures, including various methods of treatment, present a significant risk of stroke.

For example, stroke and other ischemic damage can often occur in a patient after treatment for an aneurysm, whether through a surgical or endovascular procedure. In the surgical setting, there is a clear association with ischemic brain damage and temporary arterial occlusion, with 26% of patients having evidence of stroke, as determined by magnetic resonance imaging (MRI). See Ferch et al., J. Neurosurg, 97:836-42, (2002). Likewise, endovascular treatment of aneurysms is associated with a significant rate of ischemia. See Cronqvist et al., Neuroradiology, 43:662-671 (2001); and Hadjivassiliou et al., Neurology 56:1672-1677, (2001).

Unfortunately, the prior art has primarily focused on methods of treating ischemia after the medical procedure, having a significant risk of ischemia, has already been performed. Accordingly, there is a need in the art to prevent or ameliorate the effects of ischemia, by pre-treating a susceptible patient, specifically before performing a medical procedure that is associated with a significant ischemic risk.

### SUMMARY OF THE INVENTION

Certain embodiments herein include methods of treatment, including identifying a mammalian, preferably human patient that is susceptible to ischemia; administering a sufficient amount of a nitroxide to prevent or ameliorate a harmful effect of ischemia in the human patient prior to the onset of ischemia.

Additional embodiments relate to uses of a nitroxide for the preparation of a medicament to prevent a harmful effect of ischemia in a mammalian, preferably human patient prior to the onset of ischemia.

Further embodiments include medicaments comprising nitroxide for the treatment of ischemia, wherein said treatment comprises identifying a patient that is susceptible to ischemia, and administering a sufficient amount of said medicament to prevent a harmful effect of ischemia in the patient prior to the onset of ischemia.

In specific embodiments, the nitroxide to be used with the methods, medicaments and uses herein is 4-hydroxy-2,2,6,6-tetramethylpiperidine-1-oxyl. In additional embodiments, the teachings herein can be used to treat a human patient whose susceptibility to ischemia arises from a medical procedure associated with a significant ischemic risk. In certain embodiments, these medical procedures can include the treatment of a hemorrhage, an aneurysm, a particular surgery or endovascular procedure, for example. In specific embodiments, the nitroxide can be administered orally or intravenously.

### DETAILED DESCRIPTION OF THE PREFERRED EMBODIMENT

The teachings herein generally relate to methods of pre-treating a patient who is susceptible to ischemia with a nitroxide, in order to prevent or ameliorate the negative effects of ischemia. As used herein, the term “ischemia” generally relates to physiological damage resulting from a lack of blood and oxygen flow, and encompasses strokes. As used herein, the term “stroke” relates to physiological damage resulting from a lack of blood and oxygen flow to the brain.

In certain embodiments, the methods herein can be used to prevent or ameliorate any negative effect of any type of ischemia, including ischemic and hemorrhagic strokes, for example. In an ischemic stroke, the blood supply to the brain is cut off, often because atherosclerosis or a blood clot has blocked a blood vessel. Typically ischemic stroke results from the presence of either a thrombus or an embolus. A thrombus generally relates to a clot formed within a blood vessel that remains attached to its place of origin. In contrast, an embolus generally relates to an abnormal particle, circulating in the blood. A hemorrhagic stroke occurs when a blood vessel ruptures, typically preventing normal flow.

In certain embodiments, the patient can be a human that has been identified as being susceptible to ischemia, including stroke, using any available method, including the following non-exclusive list of diagnostics: computed tomography (CT), magnetic resonance imaging (MRI, including DWI and PWI), carotid ultrasonography/doppler scanning, Magnetic resonance angiography (MRA), Carotid angiography, chest X-ray, electrocardiography (ECG, or EKG), echocardiography, Holter monitoring or telemetry, and the like, for example.

In other embodiments, susceptible patients can be identified using optical tomography, some methods of which are disclosed in U.S. Pat. No. 6,516,214, issued to Boas, which is hereby expressly incorporated by reference in its entirety.

In further embodiments, identification of susceptible patients can be based on assessing one or more available risk factors such as age, sex, race, weight, cholesterol levels, blood pressure, atherosclerosis, family history, genetic disposition, heart condition, smoking habits, consumption of alcohol, percentage of body fat, diet, diabetes, exercise, lifestyle, collagen disease, previous incidents of ischemia, including stroke, in the patient, and the like, for example.

In more specific embodiments a particular patient's susceptibility to ischemia, including stroke, can be assessed using more specific risk factors including the detection of aneurysms, coronary artery disease, including, for example, occlusions, or blocking of a patient's blood vessels. An occlusion can be partial or complete blocking of the vessel. Obstruction in blood vessels can occur as a result of a thrombus, embolus, vasospasms, arteriosclerosis, and the like, for example. Arteriosclerosis generally relates to several diseases in which the wall of an artery becomes thicker and less elastic. The most common of these diseases is atherosclerosis, in which fatty material accumulates under the inner lining of the arterial wall. Any of the above-provided conditions can be used to determine a particular patient's susceptibility to ischemia, including stroke.

Specific embodiments herein, include methods of administering a nitroxide to a patient prior to undergoing any medical procedure with a significant risk of causing a stroke or ischemia. A significant risk can include about a 1%, 5%, 10%, 15%, 20%, 25%, 30%, 35%, 40%, 45%, 50%, 55%, 60%, 65%, 70%, 75%, 80%, 85%, 90%, 95%, and 100% chance, for example.

In other embodiments a significant risk can include medical procedures where there is a greater than about a 1%, 5%, 10%, 15%, 20%, 25%, 30%, 35%, 40%, 45%, 50%, 55%, 60%, 65%, 70%, 75%, 80%, 85%, 90%, 95%, and 100% chance of ischemia, for example.

In certain embodiments, a nitroxide can be administered prior to the following non-exclusive list of medical procedures: cardiac surgery, including bypass surgery, and mitral valve surgery, carotid endarterectomy, angioplasty, craniotomy, cervical discectomy and corpectomy, cervical laminectomy, laryngectomy, parathyroidectomy, thyroidectomy, tracheostomy, hysterectomy, prostatectomy, urinary cystectomy, joint replacements (arthroplasty) including knee, shoulder, hip, ankle, wrist, and the like, for example. In further embodiments, in addition to being administered prior to a medical procedure, a nitroxide can also be administered after the medical procedure too.

In other specific embodiments, the methods provided herein include administering a nitroxide to a patient prior to undergoing surgery to treat any type of hemorrhage, such as a brain hemorrhage, for example. The term “hemorrhage” typically refers to a discharge of blood from a vessel. As used herein, the term “brain hemorrhage” non-exclusively includes intracerebral hemorrhage, subarachnoid hemorrhage, subdural hemorrhage, epidural hemorrhage, and the like, for example. In further embodiments, in addition to being administered prior to surgery to treat a hemorrhage, a nitroxide can also be administered after surgery to treat a hemorrhage.

The location of the particular hemorrhage will typically dictate which specific medical procedure a practitioner will use to treat the hemorrhage. For example, treatment for a brain hemorrhage can include placing a drainage tube in the brain to release pressure, or surgery that isolates, blocks off, or supports the weakened artery walls, and the like. While the timing of this surgery is somewhat controversial, most neurosurgeons recommend operating within 3 days of the start of symptoms. Typically, delaying the surgery 10 or more days reduces the risks of surgery but increases the chances of rebleeding in the interim. Certain embodiments provided herein include administering a nitroxide prior to conducting any of the above-described treatments for a hemorrhage.

In certain embodiments, the methods herein can include administering a nitroxide to a patient prior to undergoing any treatment for any type of aneurysm. In general, there are two basic methods of treating aneurysms, surgical and endovascular, both of which are well known in the art. A surgical procedure generally relates to an open procedure, and often involves a small vascular clip being placed across the neck of the aneurysm, thereby excluding it from the circulation. In contrast, an endovascular procedure generally relates to a closed procedure, and often involves a tiny microcatheter being navigated from the femoral artery in the groin into the blood vessels allowing the placement of specially designed coils into the dome of the aneurysm. Typically, these coils are packed into the aneurysm, filling up its volume and often preventing blood from entering. Accordingly, certain embodiments include methods of using any available nitroxide prior to a surgical or endovascular procedure to treat an aneurysm, for example. Specific embodiments include administering a nitroxide to a patient prior to undergoing any treatment for an aneurysmal subarachnoid hemorrhage, whether surgical or endovascular, for example. In further embodiments, in addition to being administered prior to surgery to treat an aneurysm, a nitroxide can also be administered after surgery to treat an aneurysm.

### Effects of Ischemia and Stroke

The methods herein include the use of a nitroxide to prevent the onset of ischemia, or to ameliorate any effect of ischemia. Typically, ischemia, including stroke, results in the generation of free radicals which participate in killing cells. These generated free radicals include reactive oxygen species (ROS), and superoxide, perhydroxyl, hydrogen peroxide, hydroxyl, and the like, for example. While not being limited by any particular mechanism of operation, the methods herein use a nitroxide to act as an antioxidant, or an ROS scavenger. Accordingly, the methods herein can prevent brain cell and tissue damage resulting from a lack of blood and oxygen. Furthermore, the methods herein involve the use of a nitroxide to prevent or ameliorate the effects that accompany brain cell and tissue damage including, but not limited to, loss of motor skills, neurologic dysfunction, infarction, formation of edemas, cellular and sub-cellular damage, including damage to organelles and molecules such as DNA and RNA, and the like, for example.

It is important to note that the methods provided herein can be used to prevent or ameliorate any type of ischemia, regardless of the particular location in the patient's body. For example, the methods herein can be used to prevent or ameliorate any effect of cardiac ischemia, myocardial ischemia, ischemia in muscle tissue, stroke, and the like. Further embodiments involve administering a sufficient amount of nitroxide prior to a patient undergoing a medical procedure associated with a significant risk of ischemia, including stroke.

### Nitroxides

The methods described herein are directed to the use of a nitroxide to prevent or ameliorate the negative effects of ischemia, including stroke, in a patient. In certain embodiments, the nitroxide can be administered to a patient that is susceptible to ischemia, including stroke. In specific embodiments, a patient's susceptibility to ischemia can arise through a medical procedure, including procedures to treat a brain hemorrhage or aneurysm, and the like, for example. Accordingly, specific embodiments include administering a prophylactic amount of a nitroxide prior to a particular medical procedure that involves a significant risk of ischemia, such as a procedure for treating an aneurysmal subarachnoid hemorrhage, for example. Other embodiments include administering a prophylactic amount of a nitroxide prior to a particular medical procedure that involves a significant risk of ischemia and then administering a therapeutic or prophylactic amount of nitroxide after the medical procedure.

As used herein the term “nitroxide” is to be construed broadly, and generally refers to stable free radical compounds that are capable of reacting with a variety of biologically relevant compounds such as free radicals, including, for example, oxy radicals. In more specific embodiments, the nitroxides described herein are free radical scavengers or anti-oxidants.

Generally nitroxides can prevent or ameliorate any effect of ischemia in a patient. These effects include, but are not limited to, oxidative stress and damage caused to healthy cells by the formation of free radicals, including necrosis and apoptosis. Furthermore, nitroxides can be used to prevent or ameliorate the effects that accompany ischemic brain cell and tissue damage including, but not limited to, loss of motor skills, neurologic dysfunction, infarction, formation of edemas, cellular and sub-cellular damage, including damage to organelles and molecules such as DNA and RNA, and the like, for example.

According to certain embodiments, the nitroxides used in the methods described herein can be selected from the following formulas:

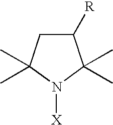

Wherein X is selected from O. and OH, and R is selected from COOH, CONH, CN, and CH₂NH₂

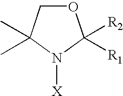

Wherein X is selected from O. and OH, and R₁ is selected from CH₃ and spirocylohexyl, and R₂ is selected from C₂H₅ and spirocyclohexyl

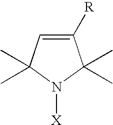

Wherein X is selected from O. and OH and R is selected from CONH.

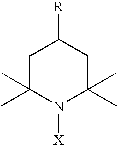

Wherein X is selected from O. and OH and R is selected from H, OH, and NH₂.

Other suitable nitroxides that can be used with the methods provided herein are found in Proctor, U.S. Pat. No. 5,352,442, and Mitchell et al., U.S. Pat. No. 5,462,946, both of which are hereby incorporated by reference in their entireties.

A non-exclusive list of nitroxides that can be used with the methods described herein also include, 2-ethyl-2,5,5-trimethyl-3-oxazolidine-1-oxyl (OXANO), 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Aminomethyl-PROXYL, 3-Cyano-PROXYL, 3-Carbamoyl-PROXYL, 3-Carboxy-PROXYL, and 4-Oxo-TEMPO.

One preferred nitroxide that can be used with the methods described herein is Tempol, characterized by the chemical formula 4-hydroxy-2,2,6,6-tetramethylpiperidine-1-oxyl. Tempol is a stable nitroxide radical that can act as a free radical scavenger to prevent or ameliorate the harmful effects of ischemia, including stroke, in a patient.

In certain embodiments, the above listed nitroxides can be used as a sole active ingredient in preventing or ameliorating any effect of ischemia in a susceptible patient. In other embodiments, the nitroxides provided above can be used with other anti-oxidants capable of neutralizing harmful free radicals generated by the onset of ischemia, including other nitroxides. Other suitable anti-oxidants that can be used in conjunction with the methods described herein include, but are not limited to: Vitamins A, B, C, and E, selenium, isoflavones, polyphenols, carotenoids, carnosines, citric acid, phenolic compounds, BHA (butylated hydroxyanisole), BHT (butylated hydroxytoluene), propyl gallate, TBHQ (tert-butyl hydroquinone), lecithins, gum or resin guiac, THBP (trihydroxybutyrophenone), thiodipropionic acid, dilauryl thiodipropionate, co-enzyme Q10, alpha-lipoic acid, anthocyanins, beta carotene, catechins, ginkgo bilboa, lutien, lycopene, glutathione and proanthocyanidins

### Methods of Using Compositions

Method embodiments include the use of any nitroxide described herein to prevent or ameliorate a negative effect in a patient resulting from ischemia. As used herein, the term “patient” generally relates to a human. In general, the term “prevent” generally relates to reducing the risk of ischemia occurring, completely preventing ischemia from occurring, and/or preventing the negative effects of ischemia, including stroke. In general, the term “ameliorate” relates to treating and/or minimizing the damage resulting from ischemia. In other embodiments, the terms “prevent” and “ameliorate” relate to an improved outcome and/or a delay of ischemia, as compared to outcomes expected or obtained in the absence of using the methods described herein.

The terms “negative effect” and “effect” are to be broadly construed, and relate to any damaging event in a patient resulting, directly or indirectly, from ischemia. These effects can include, for example, oxidative stress, necrosis, apoptosis, loss of motor skills, neurologic dysfunction, infarction, formation of edemas, cellular and sub-cellular damage, including damage to organelles, DNA and RNA, and the like, for example. In certain embodiments, the methods herein can be used prior to any currently available medical procedure having a significant risk of causing ischemia. In other embodiments, the methods herein can be used in conjunction with medical procedures, having a significant risk of ischemia, that will be developed in the future.

Method embodiments include using any nitroxide, such as those expressly described herein, on a patient who is susceptible to ischemia, including stroke, such as a patient who will be undergoing a medical procedure with a significant risk of ischemia, including stroke. In some embodiments, nitroxide, can be applied to a patient about 24, 23, 22, 21, 20, 19, 18, 17, 16, 15, 14, 13, 12, 11, 10, 9, 8, 7, 6, 5, 4, 3, or 2 hours before the patient undergoes a medical procedure with associated with a significant risk of ischemia, including stroke. In other embodiments, a nitroxide can be applied to a patient about 119, 118, 117, 116, 115, 110, 105, 100, 95, 90, 85, 80, 75, 70, 65, 60, 55, 50, 45, 40, 35, 30, 29, 28, 27, 26, 25, 24, 23, 22, 21, 20, 19, 18, 17, 16, 15, 14, 13, 12, 11, 10, 9, 8, 7, 6, 5, 4, 3, or 2 minutes before the patient undergoes a medical procedure associated with a significant risk of ischemia, including stroke. In other embodiments, a nitroxide can be applied to a patient about 119, 118, 117, 116, 115, 110, 105, 100, 95, 90, 85, 80, 75, 70, 65, 60, 55, 50, 45, 40, 35, 30, 29, 28, 27, 26, 25, 24, 23, 22, 21, 20, 19, 18, 17, 16, 15, 14, 13, 12, 11, 10, 9, 8, 7, 6, 5, 4, 3, or 2 seconds before the patient undergoes a medical procedure associated with a significant risk of ischemia, including stroke.

In other embodiments, the nitroxides provided herein can be applied on a regular basis, to a patient who has been identified as being susceptible to ischemia, including stroke, based on any available method of identification, including assessing one or more relevant risk factors, for example.

Nitroxides can be administered to a patient according to any available method, including orally, topically, or parenterally, for example, by injection. Oral administration can be in the form of tablets, solution, syrup, gel capsules, and the like, for example. Injection can be subcutaneous, intravenous, or by intramuscular injection, and the like, for example.

Any dose of a particular nitroxide that is capable of preventing or ameliorating the effects of ischemia, including stroke, can be used with the methods described herein. In certain embodiments, the nitroxide can be used at a dose of about 1, 1.5, 2, 2.5, 3, 3.25, 3.5, 3.75, 4, 4.25, 4.5, 4.75, 5, 5.25, 5.5, 5.75, 6, 6.25, 6.5, 6.75, 7, 7.25 7.5, 7.75, 8, 8.25, 8.5, 8.75, 9, 9.1, 9.2, 9.3, 9.4, 9.5, 9.6, 9.7, 9.8, 9.9 and 10 mg/kg, for example. In other embodiments the dose of the nitroxide can be about, 10.5, 11, 11.5, 12, 12.5, 13, 13.5, 14, 14.5, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, and 300 mg/kg, for example.

In some embodiments, the nitroxide can be administered in 1, 2, 3, 4, 5, 6, 7, 8, 9 or 10 doses prior to a medical procedure associated with a significant risk of ischemia. In other embodiments, the nitroxide can be administered about 1, 2, 3, 4, 5, 6, 7, 8, 9, or about 10 times daily. Specific embodiments include regular (e.g., monthly, twice monthly, weekly, twice weekly, thrice weekly, daily, twice daily, thrice daily) administration to a patient who is susceptible to ischemia, including stroke. In other embodiments, the nitroxide can be administered after about one or two times the half life of the nitroxide, for example.

### Characteristics of Nitroxide Formulations

A nitroxide to be used with the methods provided herein, can be incorporated into any suitable formulation or be used alone. The particular nitroxide formulation to be used herein will depend on the intended method of administration, whether the mode of administration is oral, parenteral, including injection, or topical, and the like, for example. In certain embodiments, a nitroxide can be administered in the form of a pharmaceutical composition in combination with pharmaceutically acceptable carriers or excipients, the proportion and nature of which can be determined by the solubility and chemical properties of the nitroxide selected, the chosen route of administration, and standard pharmaceutical practice. In other embodiments, the nitroxides described herein, while effective themselves, can be formulated and administered in the form of their pharmaceutically acceptable salts, such as for example, acid addition salts, for purposes of stability, convenience of crystallization, increased solubility and the like.

A nitroxide utilized in accordance with the teachings herein can be administered in any form or mode which makes the nitroxide bioavailable, including oral, parenteral, and topical routes, and the like, for example. A non-exclusive list of administration routes include, oral, subcutaneous, intramuscular, intravenous, transdermal, intranasal, rectal, topical, and the like, for example. One skilled in the art of preparing formulations can readily select the proper form and mode of administration depending upon the particular characteristics of the nitroxide selected, after assessing the relevant circumstances.

In certain embodiments, a nitroxide can include a carrier or one or more excipients. In more specific embodiments, the carrier or excipient can be a solid, semi-solid, or liquid material which can serve as a vehicle or medium for the nitroxide. Suitable carriers or excipients are well known in the art. In further embodiments, a nitroxide can be adapted for oral, parenteral, or topical use and can be administered to the patient in the form of tablets, capsules, suppositories, solution, suspensions, or the like.

In certain embodiments, a nitroxide can be administered orally, for example, with an inert diluent or with an edible carrier. In other embodiments, a nitroxide can be enclosed in a gelatin capsule or compressed into a tablet. For certain embodiments directed to oral administration, a nitroxide can be incorporated with excipients and used in the form of tablets, troches, capsules, elixirs, suspensions, syrups, wafers, chewing gums and the like.

In other embodiments, nitroxide-containing tablets, pills, capsules, troches and the like can also include adjuvants typically utilized in the preparation of pharmaceuticals. For example, they can include one or more of the following adjuvants: binders such as microcrystalline cellulose, gum tragacanth or gelatin; excipients such as starch or lactose, disintegrating agents such as alginic acid, corn starch and the like; lubricants such as magnesium stearate or zinc stearate; glidants such as colloidal silicon dioxide; and sweetening agents such as sucrose or saccharin can be added or a flavoring agent, such as peppermint, methyl salicylate or orange flavoring, for example. When the dosage unit form is a capsule, it can contain, in addition to materials described above, a liquid carrier such as polyethylene glycol or a fatty oil, and the like, for example.

In other embodiments, the dosage unit forms can contain other materials which modify the physical form of the dosage unit, for example, as coatings. Thus, tablets or pills can be coated with sugar, shellac, or other enteric coating agents. In other embodiments, nitroxide-containing syrup can include a sweetening agent, such as sucrose, and certain preservatives, dyes and colorings and flavors, and the like, for example.

In certain embodiments, the nitroxides to be used with the methods described herein, are solutes dissolved in a suitable solvent. In other embodiments, the nitroxides to be used with the methods described herein can be in the form of a dispersion, suspension, liquid, thickened liquid, gel, or emulsion, for example. In additional embodiments, the nitroxide formulations are in the form of a cream, lotion, ointment and the like. Detail on how to prepare the above formulations is provided in Remington's Pharmaceutical Sciences, 18^{th} ed. 1990, which is hereby incorporated by reference in its entirety.

In further embodiments, nitroxide solutions or suspensions used for parenteral, intradermal, or subcutaneous application may include a sterile diluent such as water for injection, a saline solution, a fixed oil, a polyethylene glycol, glycerine, propylene glycol, other synthetic solvents, an antibacterial agent, such as benzyl alcohol or methyl paraben, an antioxidant such as ascorbic acid or sodium bisulfite, a chelating agent such as ethylenediaminetetraacetic acid, a buffer such as an acetate, citrate or phosphate and an agent for the adjustment of tonicity such as sodium chloride or dextrose, and the like, for example. In further embodiments, the pH may be adjusted with acids or bases, such as hydrochloric acid or sodium hydroxide. Parenteral preparations may be enclosed in ampoules, syringes, multiple dose vials made of glass or plastic, and the like, for example.

Pharmaceutical compositions suitable for injection include sterile aqueous solutions or dispersions and sterile powders for the extemporaneous preparation of sterile injectable solutions, dispersions, and the like, for example. For intravenous administration, suitable carriers include physiological saline, bacteriostatic water, Cremophor ELTM (BASF, Parsippany, N.J.), phosphate buffered saline (PBS), and the like, for example. In other embodiments, the carrier can be a solvent or dispersion medium containing water, an alcohol such as ethanol, a polyol such as glycerol, propylene glycol, and liquid polyethylene glycol, suitable mixtures thereof, and the like, for example. In certain embodiments, these pharmaceutical compositions are fluid to the extent that easy syringability exists. The proper fluidity may be maintained by the use of a coating such as lecithin, or by the use of surfactants, and the like, for example. In more particular embodiments, pharmaceutical compositions for injection are preserved against the contaminating action of microorganisms, such as bacteria, fungi, and the like. Prevention of the action of microorganisms may be achieved by various antibacterial and antifungal agents such as parabens, chlorobutanol, phenol, ascorbic acid, thimerosal, and the like, for example. In certain embodiments, isotonic agents such as sugars, polyalcohols such as manitol, sorbitol, sodium chloride can be used in the nitroxide containing composition. Prolonged absorption of the injectable compositions may be brought about by including an agent which delays absorption such as aluminum monostearate, gelatin, and the like, for example.

Injectable solutions, to be used with the methods herein, can be prepared by any available processes known in the art. Detail on how to prepare injectable solutions is provided in Remington's Pharmaceutical Sciences, 18^{th} ed. 1990, which is hereby incorporated by reference in its entirety. In some embodiments, injectable solutions can be prepared by incorporating nitroxide in the desired amount in an appropriate solvent alone, or with one or more additional ingredients enumerated herein, or known in the art. In further embodiments, the solution can be filtered sterilized after dissolving the nitroxide.

In other embodiments, nitroxide containing dispersions can be prepared according to any available process. Detail on how to prepare injectable dispersions is provided in Remington's Pharmaceutical Sciences, 18^{th} ed. 1990, which is hereby incorporated by reference in its entirety. In certain embodiments, injectable dispersions can be prepared by incorporating nitroxide into a sterile vehicle containing a basic dispersion medium, alone, or with one or more additional ingredients, such as those provided herein or known in the art, for example.

### Suitable Solvents for Nitroxides

Nitroxides, such as Tempol, are readably soluble in aqueous solutions. In some embodiments, a nitroxide can be dissolved in a solvent and prepared into a formulation including gels, thickened liquids, liquids, and the like. Those skilled in the art will readily appreciate that any water miscible liquid, at appropriate levels, can be used as a solvent, including, but not limited to, glycerin, PEG's, polysorbates, and the like.

The following is a non-exclusive list of solvents that can be used for nitroxides: water, urea, alcohols and glycols. Any alcohol capable of dissolving nitroxides can be used in the formulations and methods described herein; examples include methanol, ethanol, propanol, butanol and the like. Likewise, any glycol capable of dissolving nitroxides can be used in the formulations and methods described herein; examples include ethylene glycol, propylene glycol and the like. In one preferred embodiment, the solvent not only dissolves the nitroxide, but also facilitates transdermal delivery. Thus, transdermal-delivery-facilitating agents, particular those that disrupt or solubilize components of the stratum corneum, are particularly preferred. In other embodiments, various alcohols that facilitate penetration of nitroxides into the skin can be used with the methods herein. Additional embodiments include available transdermal enhancers that allow for systemic treatment of a patient.

In certain embodiments of the invention, the concentration of the active ingredient, a nitroxide, can be at a concentration level at or near its solubility limit. For example a nitroxide can be about 80%, 81%, 82%, 83%, 84%, 85%, 86%, 87%, 88%, 89%, 90%, 91%, 92%, 93%, 94%, 95%, 96%, 97%, 98%, 99% and 100% of saturation in the solution. Embodiments also include formulations where a nitroxide is soluble enough in the solvent to promote its release at the desired rate upon application to the treated area. All of the above described solvents can be used with the solutions described herein, including gels, thickened liquids and liquids and the like.

### Other Methods to Prevent or Ameliorate the Effects of Ischemia and Stroke.

In some embodiments, the methods described herein include using a nitroxide in conjunction with one or more additional types of treatment to prevent or ameliorate the effects of ischemia, including stroke. The additional types of treatment can be applied either before, during, or after the onset of ischemia, including stroke. Additional treatments to be used in conjunction with nitroxides non-exclusively include administering oxygen, intravenous fluids, nourishment, anticoagulants, such as heparin, drugs that break up clots, such as streptokinase or tissue plasminogen activator, anti-swelling drugs such as mannitol or corticosteroids, anti-platelet drugs such as aspirin, clopidogrel bisulfate, and aspirin with dipyridamole, anti-hypertensive agents, such as labetalol and enalapril, and the like, for example. Additional treatments can also include medical procedures, such as surgical removal of blockages (e.g., endarterectomy) and angioplasty, and the like, for example.

The following example is provided for illustrative purposes only and is not to be construed as limiting upon the teachings herein.

### Example I

This example describes a clinical study to determine the effect of Tempol on the prevention of cerebral ischemia during treatment of aneurysms in human patients that have bled. Patients, having suffered aneurysmal subarachnoid hemorrhaging, undergo magnetic resonance imaging (MRI-DWI) to count the number of infarcts and measure their size. Either 1-300 mg/kg of Tempol or a placebo are orally administered to the human patients. Patients undergo treatment for subarachnoid hemorrhaging (e.g., surgical or endovascular). After surgery, 1-300 mg/kg of Tempol or a placebo are orally administered to the patients. 1-3 days post-treatment, follow up MRI-DWI is used to count and measure the size of infarcts. The number and size of the infarcts is also measured 6 weeks post-treatment using MRI-DWI. Results should show that patients who are given Tempol prior to and after treatment for aneurysmal subarachnoid hemorrhaging have fewer and smaller sized infarcts than patients who only receive placebos.

### EQUIVALENTS

The foregoing description and Example detail certain preferred embodiments of the teachings herein and describes the best mode contemplated by the inventors. It will be appreciated, however, that no matter how detailed the foregoing may appear in text, the methods of using a nitroxide to prevent or ameliorate the effects of a ischemia, including stroke, can be practiced in many ways and the teachings herein should be construed in accordance with the appended claims and any equivalents thereof. The foregoing written specification is considered to be sufficient to enable one skilled in the art to practice the embodiments described herein.

What is claimed is:

1. A method of treatment, comprising:
identifying a human patient that is susceptible to ischemia; and
reducing the likelihood of an occurrence of a harmful effect of ischemia by administering an effective amount of a stable free radical prior to the onset of ischemia;
wherein the likelihood is reduced in comparison to a human patient that was not subjected to the administering step.

2. The method of claim 1, wherein the nitroxide is 4-hydroxy-2,2,6,6-tetramethylpiperidine-1-oxyl.

3. The method of claim 1, wherein the human patient's susceptibility to ischemia arises from a medical procedure associated with a significant ischemic risk.

4. The method of claim 3, wherein the medical procedure is the treatment of a hemorrhage.

5. The method of claim 3, wherein the medical procedure is the treatment of an aneurysm.

6. The method of claim 3, wherein the medical procedure is surgery.

7. The method of claim 3, wherein the medical procedure is an endovascular procedure.

8. The method of claim 1, wherein the mode of nitroxide administration is selected from the group consisting of oral and intravenous administration.

9. A method of treatment comprising:
identifying a patient scheduled to undergo a medical procedure involving a risk of ischemia;
reducing the likelihood of an occurrence of a harmful effect of ischemia by administering to the patient, prior to the medical procedure, an effective amount of a stable free radical nitroxide;
performing the medical procedure; and
administering to the patient, an additional amount of a stable free radical nitroxide to ameliorate a harmful effect of ischemia.

10. The method of claim 9, wherein the nitroxide is 4-hydroxy-2,2,6,6-tetramethylpiperidine-1-oxyl.

11. The method of claim 9, wherein the medical procedure is the treatment of a hemorrhage.

12. The method of claim 9, wherein the medical procedure is the treatment of an aneurysm.

13. The method of claim 9, wherein the medical procedure is surgery.

14. The method of claim 9, wherein the medical procedure is an endovascular procedure.

15. The method of claim 9, wherein the mode of nitroxide administration is selected from the group consisting of oral and intravenous administration.

16. The method of claim 1 wherein the nitroxide is selected from the group consisting of 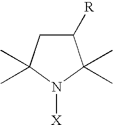
or a pharmaceutically acceptable salt thereof
wherein X is selected from O. and OH, and R is selected from COOH, CONH, CN, and CH2NH2; 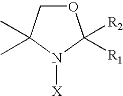
or a pharmaceutically acceptable salt thereof
wherein X is selected from O. and OH, and R₁ is selected from CH₃ and spirocylohexyl, and R₂ is selected from C₂H₅ and spirocyclohexyl; 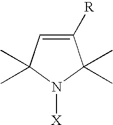
or a pharmaceutically acceptable salt thereof
wherein X is selected from O. and OH and R is CONH; 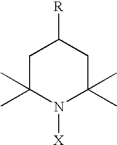
or a pharmaceutically acceptable salt thereof
wherein X is selected from O. and OH and R is H, OH, and NH₂; 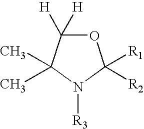
wherein R₁ is —CH₃; R₂ is —C₂H₅, —C₃H₇, —C₄H₉, —C₅H₁₁, —C₆H₁₃, —CH₂—CH(CH₃)₂, —CHCH₃C₂H₅, or —(CH₂)₇—CH₃, or wherein R₁ and R₂ together form spirocyclopentane, spirocyclohexane, spirocycloheptane, spirocyclooctane, 5-cholestane, or norbornane; R₃ is —O. or —OH, or a physiologically acceptable salt thereof which has antioxidant activity; 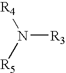
wherein R₃ is —O. or —OH; and
wherein R₄ and R₅ combine together with the nitrogen to form a heterocyclic group; wherein the atoms in the heterocyclic group (other than the N atom shown in the formula) may be all C atoms or may be C atoms and one or more N, O and/or S atoms; or
wherein R₄ and R₅ combine together to form substituted or unsubstituted pyrrole, imidazole, oxazole, thiazole, pyrazole, 3-pyrroline, pyrrolidine, pyridine, pyrimidine, or purine; or
wherein R₄ and R₅ themselves comprise a substituted or unsubstituted cyclic or heterocyclic group;
2-ethyl-2,5,5-trimethyl-3-oxazolidine-1-oxyl, 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Aminomethyl-PROXYL, 3-Cyano-PROXYL, 3-Carbamoyl-PROXYL, 3-Carboxy-PROXYL, 4-oxo-TEMPO, 4-amino-TEMPO, 4-(2-bromoacetamido)-TEMPO, 4-(ethoxyfluorophosphonyloxy)-TEMPO, 4-hydroxy-TEMPO, 4-(2-iodo acetamido)-TEMPO, 4-isothiocyanato-TEMPO, 4-maleimido-TEMPO, 4-(4-nitrobenzoyloxyl)-TEMPO, and 4-phosphonooxy-TEMPO.

17. The method of claim 9 wherein the nitroxide is selected from the group consisting of 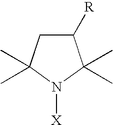
or a pharmaceutically acceptable salt thereof
wherein X is selected from O. and OH, and R is selected from COOH, CONH, CN, and CH2NH2; 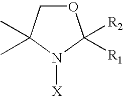
or a pharmaceutically acceptable salt thereof
wherein X is selected from O. and OH, and R₁ is selected from CH₃ and spirocylohexyl, and R₂ is selected from C₂H₅ and spirocyclohexyl; 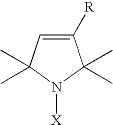
or a pharmaceutically acceptable salt thereof
wherein X is selected from O. and OH and R is CONH; 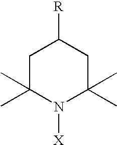
or a pharmaceutically acceptable salt thereof
wherein X is selected from O. and OH and R is selected from H, OH, and NH₂; 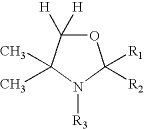
wherein R₁ is —CH₃; R₂ is —C₂H₅, —C₃H₇, —C₄H₉, —C₅H₁₁, —C₆H₁₃, —CH₂—CH(CH₃)₂, —CHCH₃C₂H₅, or —(CH₂)₇—CH₃, or wherein R₁ and R₂ together form spirocyclopentane, spirocyclohexane, spirocycloheptane, spirocyclooctane, 5-cholestane, or norbornane; R₃ is —O. or —OH, or a physiologically acceptable salt thereof which has antioxidant activity; 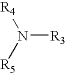
wherein R₃ is —O. or —OH; and
wherein R₄ and R₅ combine together with the nitrogen to form a heterocyclic group; wherein the atoms in the heterocyclic group (other than the N atom shown in the formula) may be all C atoms or may be C atoms and one or more N, O and/or S atoms; or
wherein R₄ and R₅ combine together to form substituted or unsubstituted pyrrole, imidazole, oxazole, thiazole, pyrazole, 3-pyrroline, pyrrolidine, pyridine, pyrimidine, or purine; or
wherein R₄ and R₅ themselves comprise a substituted or unsubstituted cyclic or heterocyclic group;
2-ethyl-2,5,5-trimethyl-3-oxazolidine-1-oxyl, 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Aminomethyl-PROXYL, 3-Cyano-PROXYL, 3-Carbamoyl-PROXYL, 3-Carboxy-PROXYL, 4-oxo-TEMPO, 4-amino-TEMPO, 4-(2-bromoacetamido)-TEMPO, 4-(ethoxyfluorophosphonyloxy)-TEMPO, 4-hydroxy-TEMPO, 4-(2-iodo acetamido)-TEMPO, 4-isothiocyanato-TEMPO, 4-maleimido-TEMPO, 4-(4-nitrobenzoyloxyl)-TEMPO, and 4-phosphonooxy-TEMPO.

18. A method of treatment comprising:
identifying a human patient who is susceptible to ischemia associated with a medical procedure; and
reducing a harmful effect of ischemia in the human patient after the medical procedure by administering an effective amount of 4-hydroxy-2,2,6,6-tetramethylpiperidine-1-oxyl prior to the onset of ischemia and prior to the medical procedure.

19. The method of claim 18, wherein the human patient's susceptibility to ischemia arises from a medical procedure associated with a significant ischemic risk.

20. The method of claim 18, wherein the medical procedure is the treatment of a hemorrhage.

21. The method of claim 18, wherein the medical procedure is the treatment of an aneurysm.

22. The method of claim 18, wherein the medical procedure is surgery.

23. The method of claim 18, wherein the medical procedure is an endovascular procedure.

24. The method of claim 18, wherein the mode of nitroxide administration is selected from the group consisting of oral and intravenous administration.

25. A method of treatment comprising:
identifying a patient scheduled to undergo a medical procedure involving a significant risk of ischemia;
reducing a harmful effect of ischemia in the human patient after the medical procedure by administering an effective amount of 4-hydroxy-2,2,6,6-tetramethylpiperidine-1-oxyl performing the medical procedure; and
administering to the patient after the performing step, an additional amount of 4-hydroxy-2,2,6,6-tetramethylpiperidine-1-oxyl effective to reduce a harmful effect of ischemia

26. The method of claim 25, wherein the nitroxide is 4-hydroxy-2,2,6,6-tetramethylpiperidine-1-oxyl.

27. The method of claim 25, wherein the medical procedure is the treatment of a hemorrhage.

28. The method of claim 25, wherein the medical procedure is the treatment of an aneurysm.

29. The method of claim 25, wherein the medical procedure is surgery.

30. The method of claim 25, wherein the medical procedure is an endovascular procedure.
